SEQUENCE LISTING (2 sequences)

SEQ ID NO: 1 - DNA, 21 bp, Artificial Sequence
  synthetic upstream primer
cactatcgactacgcgatcgg

SEQ ID NO: 2 - DNA, 24 bp, Artificial Sequence
  synthetic downstream primer
gcgctataccctataccctaaacc

(19) United States
(12) Patent Application Publication
(10) Pub. No.: US 2020/0239835 A1
(43) Pub. Date: Jul. 30, 2020

(54) METHOD FOR ISOLATING AND/OR CULTURING CAMBIUM STEM CELLS OF PANAX GINSENG

(71) Applicant: Shenzhen XianSheng Science and Technology Development Co., Ltd., Shenzhen (CN)

(72) Inventors: Dong Wu, Shenzhen (CN); Chensheng Dai, Shenzhen (CN); Lili Cheng, Shenzhen (CN); Huaide Chen, Shenzhen (CN); Minxian Liu, Shenzhen (CN); Lili Hou, Shenzhen (CN); Zhuohe Jiang, Shenzhen (CN); Yujia Liu, Shenzhen (CN); Yuanjian Ling, Shenzhen (CN)

(21) Appl. No.: 16/296,254

(22) Filed: Mar. 8, 2019

(30) Foreign Application Priority Data
Jan. 24, 2019 (CN) 201910066835.X

(51) Int. Cl. C12N 5/04

(52) U.S. Cl. CPC C12N 5/04; C12N 2500/34; C12N 2521/10; C12N 2501/39

(57) ABSTRACT

A method for isolating and/or culturing cambium stem cells of panax ginseng is disclosed. The method comprises a step of treating tissues containing panax ginseng cambium with a compound of formula I. The present application further relates to cambium stem cells of panax ginseng obtained according to the method and use of such cambium stem cells in preparing a product for a suspension culture of panax ginseng. The method for isolating and/or culturing cambium stem cells of panax ginseng according to the present application can effectively separate the cambium stem cells of the panax ginseng which have unlimited cytodieresis ability and strong anti-adversity ability, can provide a basis for ultra-large volume liquid culture, which can significantly reduce production costs.

### CROSS REFERENCE TO RELATED APPLICATION

The present application claims the benefit of Chinese Patent Application No. 201910066835.X filed on Jan. 24, 2019, the contents of which are hereby incorporated by reference.

### REFERENCE TO SEQUENCE LISTING

The Sequence Listing is submitted concurrently with the specification as an ASCII formatted text file via EFS-Web, with a file name of “Sequence_Listing.TXT”, a creation date of Aug. 6, 2019, and a size of 722 bytes. The Sequence Listing filed via EFS-Web is part of the specification and is incorporated in its entirety by reference herein.

### TECHNICAL FIELD

The present disclosure relates generally to a method for isolating and/or culturing cambium stem cells of panax ginseng, cambium stem cells obtained therefrom, and use of such cambium stem cells in preparing a product for a suspension culture of panax ginseng.

### BACKGROUND

Panax ginseng, a perennial dicotyledonous panax plant (Araliaceae), is a traditional rare Chinese herbal medicine. Currently, panax ginseng has been widely used in the fields of medicines, health products and cosmetics. Sources of the panax ginseng include wild pick, artificial cultivation, and tissue culture. Among them, the panax ginseng from natural wild sources is limited, and cannot satisfy the market demand, so the artificial cultivation is still the main source of the panax ginseng. However, the artificial cultivation has many obvious shortcomings, such as deforestation, occupation of cultivated land, long growth cycle, vulnerability to pests and diseases, pesticides and heavy metal residues, and so on. The tissue culture method obtaining the panax ginseng by culturing callus or adventitious roots can overcome the problem of limited wild source and above mentioned shortcomings of the artificial cultivation, but would not be restricted by the external environment and can obtain the main components of plants under the best conditions, which makes it the most promising source of the panax ginseng. However, callus and adventitious roots have defects such as limited cytodieresis ability, vulnerability to degeneration, and weak anti-adversity ability and so on, so they are not suitable for a large-scale continuous culture.

It has been found that plant stem cells have unlimited self-renewal ability and can differentiate into a variety of cell and tissue types. Studies have shown that when plant stem cells are embedded in the stem apical meristem, the root apical meristem, or the vascular cambium meristem, they can self-renew to divide and generate new cells, in which some of the sub-cells differentiate and the others forms new stem cells. The plant stem cells function throughout the life of the plant and provide a new cell source for the formation and regeneration of organs such as roots, stems and leaves. The vascular cambium system of the plant contains pluripotent stem cells, provides ecological niches for them and maintains their amounts and functions. In order to solve the problems of the long-term culture instability and low secondary metabolite production output due to the loss of genetic information in dedifferentiated cells, scientists have been working on the isolation and in vitro culture of plant stem cells for decades. Therefore, tissue culture from plant stem cells has become an important channel for providing valuable sources of medicinal plants.

Compared with dedifferentiated cells, the plant stem cells can retain the full set of maternal genetic information which is beneficial to the long-term stable culture, can have a lot of small vacuoles which give a strong shear resistance ability and maintain a synchronous growth as well as a high growth rate, and can synthesize various types of active secondary metabolites over a long period. However, the reported methods for isolating and/or culturing cambium stem cells of panax ginseng still cannot obtain cambium stem cells of panax ginseng with high cleavage activity and satisfied proliferation ability.

Accordingly, in order to obtain more efficient cambium stem cells of panax ginseng, improve the efficiency of tissue culture, and overcome the shortcomings of the existing methods, there is still an urgent demand for a new and more efficient method for isolating and/or culturing cambium stem cells of panax ginseng.

### SUMMARY

The object of the present application is to provide a method for isolating and/or culturing cambium stem cells of panax ginseng, capable of improving the telomerase activity.

After extensive researches, the inventor unexpectedly and surprisingly finds that when a saponin compound of formula I is used for treating tissues containing panax ginseng cambium, the telomerase activity can be significantly improved:

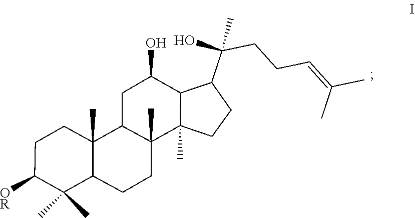

wherein R can be -β-D-glucopyranosyl(1-2)-β-D-glucopyranose (saponin a) or -β-D-glucopyranose (saponin b).

Based on above finding, in one aspect, a method for isolating and/or culturing cambium stem cells of panax ginseng is provided, comprising a step of treating tissues containing panax ginseng cambium with a compound of formula I:

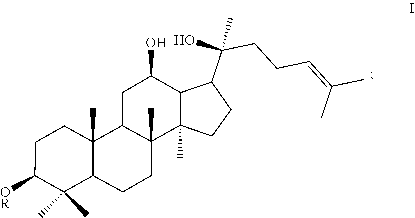

wherein R is -β-D-glucopyranosyl(1-2)-β-D-glucopyranose or -β-D-glucopyranose.

Preferably, in the method for isolating and/or culturing cambium stem cells of panax ginseng according to the present application, the tissues containing panax ginseng cambium is obtained by following step: exfoliating tissues containing cambium, phloem, cortex and epidermis off from xylogen.

In some preferred embodiments of the present application, the step of treating tissues containing panax ginseng cambium with the compound comprises placing the tissues containing panax ginseng cambium into a solution containing the compound of formula I. Wherein, the solution containing the compound of formula I is preferably an aqueous solution of the compound of formula I.

In some embodiments of the present application, the compound of formula I for treating tissues containing panax ginseng cambium can be one compound, such as the compound of formula I with R as -β-D-glucopyranosyl(1-2)-β-D-glucopyranose (saponin a) or the compound of formula I with R as -β-D-glucopyranose (saponin b); or can be a mixture of saponin a and saponin b, that is, a mixture of the compound of formula I with R as -β-D-glucopyranosyl(1-2)-β-D-glucopyranose and the compound of formula I with R as -β-D-glucopyranose with any molar ratio. In some more preferable embodiments of the present application, a molar ratio of the compound of formula I with R as -β-D-glucopyranosyl(1-2)-β-D-glucopyranose (saponin a) and the compound of formula I with R as -β-D-glucopyranose (saponin b) can be from 1:10 to 10:1, preferably from 1:5 to 5:1, more preferably from 1:3 to 3:1. Particularly preferably, the molar ratio of saponin a and saponin b is 2:5.

In the method for isolating and/or culturing cambium stem cells of panax ginseng according to the present application, the solution containing the compound of formula I has a concentration from 1 μM to 100 μM, preferably from 10 μM to 100 μM. In a particularly preferred embodiment, the compound of formula I is a mixture of saponin a and saponin b with a molar ratio of 2:5, wherein the saponin a has a concentration of 20 μM in the solution, while the saponin b has a concentration of 50 μM in the solution, respectively.

In some preferred embodiments of the present application, an ultrasonic treatment is preformed on the tissues containing panax ginseng cambium after the treatment of the compound of formula I. Preferably, the ultrasonic treatment has a treatment frequency from 5 kHz to 100 kHz, preferably from 20 kHz to 40 kHz, and a treatment time from 0.1 min to 10 min.

In some preferred embodiments of the present application, after the treatment of the compound of formula I and/or the ultrasonic treatment, the tissues containing panax ginseng cambium is further treated by a sucrose solution.

In some preferred embodiments of the present application, the method for isolating and/or culturing cambium stem cells of panax ginseng according to the present application can comprise following steps:

(1) a sterilization step comprising sterilizing washed panax ginseng medicinal materials with a sterilizing agent;

(2) an anti-browning treatment step comprising treating sterilized panax ginseng medicinal materials with an anti-browning culture medium containing an antioxidant;

(3) a separation step comprising placing panax ginseng medicinal materials after the anti-browning treatment into a cutting fluid containing an antioxidant, and exfoliating tissues containing cambium, phloem, cortex and epidermis off from xylogen;

(4) a saponin treatment step comprising treating exfoliated tissues with the saponin a and/or saponin b according to the present application, and implementing an optional ultrasound treatment or sucrose treatment;

(5) a culture step comprising culturing the tissues after the saponin treatment, and then separating the same for obtaining cambium stem cells.

In the method for isolating and/or culturing cambium stem cells of panax ginseng according to the present application, the sterilization in step (1) preferably comprises implementing a surface sterilization with 75% ethanol and then a sterilization with sodium hypochlorite preferably having a concentration of 2% for more preferably two times comprising a first preferable sterilization time of 8 min, and a second sterilization time of 4 min.

In the method for isolating and/or culturing cambium stem cells of panax ginseng according to the present application, the ultrasound treatment in step (4) preferably has a treatment frequency of 20 kHz, and a treatment time of 5 min; the sucrose treatment in step (4) preferably has a treatment concentration of 1 M and a treatment time of 4 h. The saponin treatment preferably has a treatment concentration of 20 uM and a treatment time of 5 h when the saponin a is employed, while has a treatment concentration of 50 uM and a treatment time of 5 h when the saponin b is employed.

In the method for isolating and/or culturing cambium stem cells of panax ginseng according to the present application, the culturing in step (5) preferably comprises a preliminary culturing preferably by a MS culture medium or a B5 culture medium, and a followed sub-culturing preferably by a MS culture medium. The B5 culture medium preferably comprises 3.0 mg/L IBA and 0.5 mg/L KT. The MS culture preferably comprises 3.0 mg/L 2, 4-dichlorophenoxyacetic acid (2,4-D) and 6.0 mg/L NAA.

In another aspect of the present application, cambium stem cells of panax ginseng obtained according to the above methods are provided.

In a further aspect of the present application, use of such cambium stem cells of panax ginseng obtained according to the above methods in preparing a product for a suspension culture of panax ginseng, is also provided. For example, the cambium stem cells of panax ginseng obtained by the present application can be used for suspension culture to obtain a panax ginseng plant.

The beneficial effects brought by the technical solutions provided by the embodiments of the present application are as follows. The method for isolating and/or culturing cambium stem cells of panax ginseng according to the present application can effectively separate the cambium stem cells of the panax ginseng which have unlimited cytodieresis ability and strong anti-adversity ability, can grow quickly and improve the telomerase activity. Moreover, the specific tissue can be effectively made necrosis by the ultrasonic treatment and the hypertonic treatment. The cambium stem cells can be effectively induced due to their strong anti-reverse ability, and the time of hypertonic treatment is effectively shortened, and the probability of bacteria infection is reduced. At the same time, the hormone concentration in the culture medium is controlled so that the somatocyte would not be proliferated while cambium stem cells are proliferated. Moreover, the anti-browning treatment will reduce the browning of cells during the culture.

### DETAILED DESCRIPTION OF THE PREFERRED EMBODIMENT

### Embodiment 1 Method of Isolating and Culturing

(1) Cleaning and Sterilization

The healthy, unbroken panax ginseng roots are rinsed with tap water for 30 minutes, then placed in a sterilized flask on an ultra-clean bench for 1 minute with 75% ethanol, and then rinsed for 3 to 5 times with sterile distilled water. The sterilized panax ginseng roots are further sterilized with 0.5% to 10% sodium hypochlorite solution for 5 to 10 minutes, then the sterilizing agent is discarded. Then the panax ginseng roots are rinsed with sterile distilled water for 3 to 5 times. After that, the 0.5% to 10% sodium hypochlorite solution is further used to sterilize for 3 to 5 minutes, and then the sterilizing agent is discarded. Finally, the treated panax ginseng roots are rinsed with sterile distilled water for 3 to 5 times.

(2) Anti-Browning Treatment Step

The above-mentioned sterilized panax ginseng roots are paced in the anti-browning culture medium containing an antioxidant (referring Table 1 below), and a shake flask culture is carried out for about 30 minutes to 1 hour. The sterilized filter paper is then used to remove moisture from the panax ginseng roots.

TABLE 1
Compositions of anti-browning culture medium

| Composition | Content | |
|---|---|---|
| WPM culture medium | ¼ salt content | |
| Sucrose | 1% | (w/v) |
| Polyvinyl Pyrrolidone | 0.5% | (w/v) |
| Ascorbic acid | 100 | mg/L |
| Citric acid | 150 | mg/L |

pH 5.8

(3) Separation

The panax ginseng roots after the sterilization and the anti-browning treatment are placed into the sterilization plate filled with the cutting fluid containing the antioxidant (ascorbic acid), and then the tissues containing the cambium, phloem, cortex and epidermis are gently cut off from the xylem with a sterile scalpel, and then exfoliated off, and the exfoliated tissues are inoculated into WPM preculture medium for 30 min.

TABLE 2
Compositions of cutting fluid

| Composition | Content |
|---|---|
| Polyvinyl Pyrrolidone | 0.5% (w/v) |
| Ascorbic acid | 100 mg/L |
| Citric acid | 150 mg/L |

(4) Saponin Treatment

The precultured exfoliated tissues are placed into an aqueous solution containing a saponin compound (a mixture of saponin a and saponin b in a molar ratio of 2:5 and having a concentration of 20 μM and 50 μM in the solution respectively) for 5 min. The exfoliated tissues are placed in 1 M sucrose aqueous solution, and firstly treated with ultrasonic waves at a frequency of 20 kHz and a power of 20 W for 5 min, and then subjected to a low temperature treatment for 4 hours. After that, the exfoliated tissue after the ultrasonic treatment are placed in 0.05 M sucrose aqueous solution for 5 min, and finally in 0.1 M sucrose aqueous solution for 5 min. Then the solution is aspirated with a sterile pipette and the sucrose is also removed, then the specific tissues (phloem, xylem, pith, etc.) are necrotic and only the cambium (metaphase tissue) is induced.

(5) Culture

The tissue obtained after the above treatment is inoculated to a B5 culture medium containing 30 g/L sucrose, 0.7 g/L agarose, 3.0 mg/L 2,4-dichlorophenoxyacetic acid, 3.0 mg/L IBA and 0.5 mg/L KT, and cultured in the dark at 20° C.

After two weeks of culture, the explants with obvious cambium proliferation are taken out, and the cambium stem cells are separated and transferred to a subculture medium of MS culture medium containing 30 g/L sucrose, 3.0 mg/L 2,4-dichlorophenoxyacetic acid, and 6.0 mg/L NAA. The subculture is carried out for once every two weeks, a large number of cambium stem cells are obtained in a short period of time.

### Embodiment 2 Telomerase Activity Detection

Detection Method of the Telomerase Activity

Objective: Telomerase activity in cell clusters obtained under different treatment conditions is detected, and the effects of different treatment conditions on telomerase activity are compared.

Telomerase Detection Steps:

1. Extraction of Telomerase: 1 g vigorously growing panax ginseng cell clusters are ground into uniform powders by adding liquid nitrogen, and then transferred rapidly to 50 mL centrifugal tube. 10 ml pre-cooled lysate (Tris-HCl, pH 7.4, 50 mM; MgCl₂ 15 mM; KCl 1M; EGTA 0.25M; DTT 0.1M; PMSF 12 mM; PVP 7.5%; glyceride 50%; DEPC treated water for constant volume) is added for treating, then the mixture is incubated in an ice bath and oscillated for 5 min at 4° C., 16000×g, and then centrifuged for 20 min. After that, the supernatant is transferred to a centrifuge tube, in which 4% (v/v) PEG6000 is added. Then the mixture is incubated in an ice bath at 100 rpm for 30 min to mix uniformly. After that, the mixtures are subpackaged in 2 ml centrifugal tubes to be centrifuged at 16000×g for 15 min, and then the supernatant is removed. Lysate of ¼ original amount is added to the precipitate for resuspending and lysing again. The obtained product is incubated in an ice bath at 4° C., 100 rpm for 30 min and then centrifuged at 16000×g for 2 min. After that, the supernatant is taken out, and RNase inhibitor (40 U/ul) is added in the supernatant. The extracted proteins are stored at −20° C. and waiting for use.

2. Telomerase enzymatic reaction: Telomerase DNA fragments are synthesized by the reverse transcription of the enzymatic reaction of the extracted proteins. The enzymatic reaction liquid comprises Tris-HCl (pH 8.3) 15 μM, KCl 15 μM, EGTA 3 μM, MgCl2 1.5 μM, BSA 0.01%, dNTP 0.015 μM, Triton x-100 0.01%, DTT 0.3 μM, primers 0.36 μM, protein extracts of proper amount in 300 μL system. Among them, the upstream primer is GG: CACTATCGACTACGCGATCGG (SEQ ID NO: 1), 21 bp, and the downstream primer is ACX: GCGCTATACCCTATACCCTAAACC (SEQ ID NO: 2), 24 bp.

3. Telomerase activity detection by TRAP method: The reaction system comprises rTaq enzyme 25 μL, primer 2 μL, enzymatic reaction liquid 15 μL, ddH₂O 8 μL. The PCR procedure parameters are 95° C. 5 min, 95° C. 30 sec, 47° C. 30 sec, 72° C. 40 sec, 30 circles. The PCR products are collected by ethanol precipitation method, and 12% polyacrylamide gel electrophoresis is implemented. The obtained electrophoresis strip is dyed by the silver staining method and then the telomerase activity is judged according to the number of strips.

The results shows that there is no significant change in the activity of telomerase when it is treated with ginsenoside of low concentration, but the activity of telomerase decreases when the concentration of ginsenoside is too high, for example, higher than 200 μM. The results show that the optimum concentration is about 10 μM to 100 μM. When the saponin is a mixture of the saponin a and saponin b, it is preferably a mixture of saponin a and saponin b with a molar ratio of 2:5, and the optimum concentrations of saponin a and saponin b are 20 μM and 50 μM, respectively.

The method for isolating and/or culturing cambium stem cells of panax ginseng according to the present application can effectively separate the cambium stem cells of the panax ginseng which have unlimited cytodieresis ability and strong anti-adversity ability, can provide a basis for ultra-large volume liquid culture, which can significantly reduce production costs. Moreover, the specific tissue can be effectively made necrosis by the ultrasonic treatment and the hypertonic treatment. The cambium stem cells can be effectively induced due to their strong anti-reverse ability, and the time of hypertonic treatment is effectively shortened, and the probability of bacteria infection is reduced. At the same time, the hormone concentration in the culture medium is controlled so that the somatocyte would not be proliferated while cambium stem cells are proliferated. Moreover, the anti-browning treatment will reduce the browning of cells during the culture.

### Embodiment 3 Effect Experiments of Treatment and Hypertonic Time on Induction Rate of Cambium Stem Cells of Panax Ginseng

Taking the ultrasound frequency, treatment time and hyperosmotic treatment time as the influencing factors, orthogonal experiment analysis of stem cell induction rate is carried out to explore the best treatment method for improving the induction efficiency of the cambium stem cells of panax ginseng and reducing the bacteria infection risk. In these embodiments, the saponin compound used for treatment is a mixture of the saponin a and saponin b with a molar ratio of 2:5 and the optimum concentrations of 20 μM and 50 μM, respectively.

TABLE 3
Orthogonal experiment factor level table

| | factors | | |
|---|---|---|---|
| | A Ultrasound | B Ultrasound | C Hyperosmotic |
| level | frequency (kHz) | time (min) | treatment time (h) |
| 1 | 20 | 0 | 2 |
| 2 | 30 | 5 | 4 |
| 3 | 40 | 10 | 6 |

The experiment results are listed in table 4.

TABLE 4
Experiment solution and experiment data analysis table

| | A | B | C | |
|---|---|---|---|---|
| Exper- | Ultrasound | Ultra- | Hyperosmotic | Experiment results |
| iment | frequen- | sound | treatment | (* Stem cell |
| Number | cy (kHz) | time (min) | time (h) | induction rate %) |
| 1 | 20 | 0 | 2 | 25.6 |
| 2 | 20 | 5 | 4 | 95.2 |
| 3 | 20 | 10 | 6 | 89.4 |
| 4 | 30 | 0 | 4 | 63.3 |
| 5 | 30 | 5 | 6 | 87.7 |
| 6 | 30 | 10 | 2 | 58.9 |
| 7 | 40 | 0 | 6 | 51.5 |
| 8 | 40 | 5 | 2 | 35.1 |
| 9 | 40 | 10 | 4 | 32.3 |
| K₁ | 210.2 | 140.4 | 119.6 | |
| K₂ | 209.9 | 218.0 | 190.8 | |
| K₃ | 145.5 | 180.6 | 228.6 | |
| k₁ | 70.1 | 46.8 | 39.9 | |
| k₂ | 70.0 | 72.7 | 63.3 | |
| K₃ | 48.5 | 60.2 | 76.2 | |
| R | 21.6 | 25.9 | 36.3 | |

Notes:
* Stem cell induction rate means that just the loose stem cells are inducted, no obvious callus cells, no infection of bacteria.

It can be seen from the experimental data that appropriate increase of ultrasonic frequency and ultrasonic time can effectively shorten the hyperosmotic treatment time, however excessively high ultrasonic frequency or excessively long ultrasound time will decrease the induction rate of cambium stem cells. The preferred treatment parameters comprises: ultrasonic frequency 20 kHz, ultrasonic treatment time 5 min, saponin a with a concentration of 20 μM and saponin b with a concentration of 50 μM, respectively, 1 M sucrose hypertonic treatment time 4 h.

The above are only the preferred embodiments of the present application, and are not intended to limit the scope of the present application. Any modifications, equivalents, improvements, etc., which are within the spirit and scope of the present application, should be included in the protection of the present application.

What is claimed is:

1. A method for isolating and/or culturing cambium stem cells of panax ginseng comprising a step of treating tissues containing panax ginseng cambium with a compound of formula I: 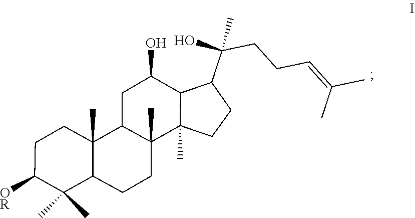
wherein R is -β-D-glucopyranosyl(1-2)-β-D-glucopyranose or -β-D-glucopyranose.

2. The method according to claim 1, wherein the tissues containing panax ginseng cambium is obtained by following step: exfoliating tissues containing cambium, phloem, cortex and epidermis off from xylogen.

3. The method according to claim 1, wherein the step of treating tissues containing panax ginseng cambium with the compound comprises placing the tissues containing panax ginseng cambium into a solution containing the compound of formula I.

4. The method according to claim 2, wherein the step of treating tissues containing panax ginseng cambium with the compound comprises placing the tissues containing panax ginseng cambium into a solution containing the compound of formula I.

5. The method according to claim 4, wherein the solution containing the compound of formula I has a concentration from 1 μM to 100 μM.

6. The method according to claim 1, wherein the compound of formula I is a mixture of the compound of formula I with R as -β-D-glucopyranosyl(1-2)-β-D-glucopyranose (saponin a) and the compound of formula I with R as -β-D-glucopyranose (saponin b) with a molar ratio of 2:5.

7. The method according to claim 5, wherein the compound of formula I is a mixture of the compound of formula I with R as -β-D-glucopyranosyl(1-2)-β-D-glucopyranose (saponin a) and the compound of formula I with R as -β-D-glucopyranose (saponin b) with a molar ratio of 2:5.

8. The method according to claim 1, wherein further comprising performing an ultrasonic treatment on the tissues containing panax ginseng cambium after the treatment of the compound of formula I.

9. The method according to claim 7, wherein further comprising performing an ultrasonic treatment on the tissues containing panax ginseng cambium after the treatment of the compound of formula I.

10. The method according to claim 9, wherein the ultrasonic treatment has a treatment frequency from 20 kHz to 40 kHz, and a treatment time from 0.1 min to 10 min.

11. The method according to claim 1, wherein further comprising treating the tissues containing panax ginseng cambium by a sucrose solution after the treatment of the compound of formula I and/or an ultrasonic treatment.

12. The method according to claim 7, wherein further comprising treating the tissues containing panax ginseng cambium by a sucrose solution after the treatment of the compound of formula I and/or the ultrasonic treatment.

13. A product comprising cambium stem cells of panax ginseng obtained according to the method according to claim 1.

14. The product according to claim 13, wherein the tissues containing panax ginseng cambium is obtained by following step: exfoliating tissues containing cambium, phloem, cortex and epidermis off from xylogen.

15. The product according to claim 13, wherein the step of treating tissues containing panax ginseng cambium with the compound comprises placing the tissues containing panax ginseng cambium into a solution containing the compound of formula I.

16. The product according to claim 15, wherein the solution containing the compound of formula I has a concentration from 1 μM to 100 μM.

17. The product according to claim 13, wherein the compound of formula I is a mixture of the compound of formula I with R as -β-D-glucopyranosyl(1-2)-β-D-glucopyranose (saponin a) and the compound of formula I with R as -β-D-glucopyranose (saponin b) with a molar ratio of 2:5.

18. The product according to claim 13, wherein an ultrasonic treatment is preformed on the tissues containing panax ginseng cambium after the treatment of the compound of formula I.

19. The product according to claim 13, wherein after the treatment of the compound of formula I and/or an ultrasonic treatment, the tissues containing panax ginseng cambium is further treated by a sucrose solution.

20. Use of the product according to claim 13 in preparing a product for a suspension culture of panax ginseng.
